(19) United States
(12) Patent Application Publication
(10) Pub. No.: US 2017/0357752 A1
(43) Pub. Date: Dec. 14, 2017

(54) SYSTEMS AND METHODS FOR AUTOMATED ANNOTATION AND SCREENING OF BIOLOGICAL SEQUENCES

(71) Applicant: TWIST BIOSCIENCE CORPORATION, San Francisco, CA (US)

(72) Inventor: James DIGGANS, San Carlos, CA (US)

(21) Appl. No.: 15/619,322

(22) Filed: Jun. 9, 2017

Related U.S. Application Data
(60) Provisional application No. 62/348,786, filed on Jun. 10, 2016.
(60) Provisional application No. 62/375,858, filed on Aug. 16, 2016.

(51) Int. Cl. G06F 19/22; G06F 19/28; C12N 15/10

(52) U.S. Cl. CPC G06F 19/22; C12N 15/1068; G06F 19/28

(57) ABSTRACT

The present disclosure describes software tools for effective biosecurity based on community knowledge and participation. Annotation tools described herein provide assistance to the synthetic biology community to track emerging science on the link between individual proteins and negative outcomes. Screening tools described herein enables the community to broaden both interest and effective practice of biosecurity so that practitioners and biological sequence or construct providers are empowered to evaluate the safety of order requests rather than waiting until synthesis or even expression. In addition, screening tools described herein provide for screening of polynucleotides across the same or multiple orders for sequences associated with harmful biological sequences from a reference database.

### CROSS-REFERENCE

This application claims the benefit of U.S. provisional patent application No. 62/348,786 filed on Jun. 10, 2016 and U.S. provisional patent application No. 62/375,858 filed on Aug. 16, 2016, each of which is incorporated by reference in its entirety.

### BACKGROUND

The growth rate in our collective knowledge about individual proteins and biological systems capable of posing potential threats to public safety and/or the environment is tremendous. This knowledge, however, is widely distributed across diverse research communities, institutions and even journals. There is a lack of centralized information source focused on annotating the potential for a given protein to cause harm and in what context this harm can arise. Thus, new systems and methods are necessary to address the challenge.

### BRIEF SUMMARY

Provided herein are computerized systems for providing enhanced polynucleotide synthesis comprising a server for hosting a database, wherein the database is adapted for representing a list of harmful biological sequences; a network connection; and a computer readable medium comprising instructions for a general purpose computer, wherein said computerized system is configured for operating in a method of: 1) receiving one or more design instructions, wherein the design instructions comprise a plurality of biological sequences, wherein each of the biological sequences is no more than 500 bases in length, and wherein the plurality of biological sequences comprise a nucleic acid or amino acid sequence; 2) automatically determining whether at least two of the plurality of biological sequences collectively corresponds to at least 20% of a harmful biological sequence in the database; and 3) automatically generating an alert if at least 20% of the harmful biological sequence is detected. Further provided herein are computerized systems further comprising wherein if no alert is generated, then one or more sequences are synthesized. Further provided herein are computerized systems further comprising receiving instructions for changing the at least two of the plurality of biological sequences corresponding to at least 20% of the harmful biological sequence to remove the harmful biological sequence. Further provided herein are computerized systems wherein the plurality of received design instructions are received at a one or more time points. Further provided herein are computerized systems wherein the plurality of received design instructions are from 3 or more different sources. Further provided herein are computerized systems wherein the plurality of received design instructions are from 5 or more different sources. Further provided herein are computerized systems wherein the plurality of received design instructions are from 10 or more different sources. Further provided herein are computerized systems wherein the one or more biological sequences are each no more than 200 bases in length. Further provided herein are computerized systems wherein the one or more biological sequences are each no more than 100 bases in length. Further provided herein are computerized systems wherein the one or more biological sequences are each no more than 50 bases in length. Further provided herein are computerized systems wherein the one or more biological sequences are each no more than 20 bases in length.

Provided herein are methods for providing enhanced polynucleotide synthesis comprising: 1) receiving one or more design instructions, wherein the design instructions comprise a plurality of biological sequences, wherein each of the biological sequences is no more than 500 bases in length, and wherein the plurality of biological sequences comprise a nucleic acid or amino acid sequence; 2) automatically determining whether at least two of the plurality of biological sequences collectively corresponds to at least 20% of a harmful biological sequence in the database; and 3) automatically generating an alert if at least 20% of the harmful biological sequence is detected. Further provided herein are methods further comprising wherein if no alert is generated, the one or more sequences are synthesized. Further provided herein are methods further comprising receiving instructions for changing the at least two of the plurality of biological sequences corresponding to at least 20% of the harmful biological sequence to remove the harmful biological sequence.

Provided herein are computerized systems for providing enhanced polynucleotide synthesis comprising a server for hosting a database, wherein the database is adapted for representing a list of sequences; a network connection; and a computer readable medium comprising instructions for a general purpose computer, wherein said computerized system is configured for operating in a method of: 1) receiving one or more design instructions, wherein the design instructions comprise a plurality of biological sequences, wherein the plurality of biological sequences is a vector sequence, and a plurality of additional insert sequences; 2) automatically determining whether the vector and at least a one of the plurality of insert sequences collectively corresponds to at least 20% of a harmful biological sequence in the database; and 3) automatically generating an alert if at least 20% of the harmful biological sequence is detected. Further provided herein are computerized systems wherein the biological sequences are obtained from sequencing a physical nucleic acid sample. Further provided herein are computerized systems further comprising wherein if no alert is generated, the one or more biological sequences are synthesized. Further provided herein are computerized systems further comprising receiving instructions for changing the at least two of the plurality of biological sequences corresponding to at least 20% of the harmful biological sequence to remove the harmful biological sequence. Further provided herein are computerized systems for providing enhanced polynucleotide synthesis wherein the plurality of received design instructions are received at one or more time points. Further provided herein are computerized systems wherein the plurality of received design instructions are received from different sources. Further provided herein are computerized systems wherein the plurality of received design instructions are from 3 or more different sources. Further provided herein are computerized systems wherein the plurality of received design instructions are from 5 or more different sources. Further provided herein are computerized systems wherein the plurality of received design instructions are from 10 or more different sources. Further provided herein are computerized systems wherein the one or more biological sequences are each no more than 200 bases in length. Further provided herein are computerized systems wherein the one or more biological sequences are each no more than 100 bases in length. Further provided herein are computerized systems wherein the one or more biological sequences are each no more than 50 bases in length. Further provided herein are computerized systems wherein the one or more biological sequences are each no more than 20 bases in length.

Provided herein are methods for providing enhanced polynucleotide synthesis comprising: 1) receiving one or more design instructions, wherein the design instructions comprise a plurality of biological sequences, wherein the plurality of biological sequences is a vector sequence, and a plurality of additional insert sequences; 2) automatically determining whether the vector and at least a one of the plurality of insert sequences collectively corresponds to at least 20% of a harmful biological sequence in the database; and 3) automatically generating an alert if at least 20% of the harmful biological sequence is detected. Further provided herein are methods wherein the biological sequences are obtained from sequencing a physical nucleic acid or protein sample. Further provided herein are methods further comprising wherein if no alert is generated, the one or more biological sequences are synthesized. Further provided herein are methods further comprising receiving instructions for changing the at least two of the plurality of biological sequences corresponding to at least 20% of the harmful biological sequence to remove the harmful biological sequence.

### INCORPORATION BY REFERENCE

All publications, patents, and patent applications mentioned in this specification are herein incorporated by reference to the same extent as if each individual publication, patent, or patent application was specifically and individually indicated to be incorporated by reference in their entirety.

### BRIEF DESCRIPTION OF THE DRAWINGS

The technical features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of the following.

FIG. 1 illustrates a user interface which includes a protein sequence and associated species, host, pathogen, route to harm, outcome and protein type information. Also included are sequence accession number, a listing of identical proteins, links to a database with sequence records, and links to similar proteins.

FIG. 2 illustrates a user interface which includes a partial listing of protein variants and an exemplary protein, “Hemagglutinin Neuraminidase-Newcastle Disease virus.”

FIG. 3A depicts a flow chart including information from a query file, a protein database, a blast report, restricted lists (harmful sequence lists) and screen report.

FIG. 3B depicts a flow chart which includes various forms of input (nucleic acid material, nucleic acid or protein sequence), decision making (restricted list, unrestricted list, expert review), and output (issuing alerts).

FIG. 4 illustrates a user interface which includes lists of databases for searching in a screen. Columns for role, type, name, description, date added and active state columns are included.

FIG. 5 illustrates a user interface which includes a sequence submission screen. Form entries for name, database, description and fasta file, and a “Submit” button are included. The database form has a drop-down column that appears upon click with subcategories, including “Seqshield,” “nr” and “Personal Database.”

FIG. 6 illustrates a user interface which includes a summary of screening status.

FIG. 7 illustrates a user interface which includes a pull-down menu for selection of “Unreviewed,” “Of concern,” or “No concern” sequences screened.

FIG. 8 illustrates a computing system.

FIG. 9 illustrates a computer system.

FIG. 10 is a block diagram illustrating an architecture of a computer system.

FIG. 11 is a diagram demonstrating a network configured to incorporate a plurality of computer systems, a plurality of cell phones and personal data assistants, and Network Attached Storage (NAS).

FIG. 12 is a block diagram of a multiprocessor computer system using a shared virtual address memory space.

### DETAILED DESCRIPTION

With the rapid growth in design capability in synthetic biology, it is now possible to create large numbers of constructs often using a heavily mutated sequence that does not directly resemble the reference sequence from which it was originally derived. At the same time, scientific advances in the understanding of the processes behind pathogenicity (in a variety of hosts and biological contexts) are rapidly creating new knowledge of protein sequences that, in context-dependent ways, can cause harm to human beings, specific plants or animals, or to the environment more broadly.

Ethical, responsible synthetic biologists may unwittingly create constructs capable of causing harm, but be unable to predict or understand that capability prior to instantiating synthetic designs in living systems. As predicting function from primary sequence alone is not feasible, these scientists would be well-served by having access to 1) a repository of metadata on what sequences can cause harm along with regulatory status and 2) an effective screening system for checking DNA or protein sequences against that metadata and alerting the user to any potential concern. In addition, a screening system capable of addressing these needs must itself be amenable to automation so as to fit seamlessly into high-throughput design/build/test workflows. The present disclosure provides for software tools to address both the lack of publicly available gene-level metadata on pathogenicity as well as the lack of open source tools for effective screening.

### Definitions

While various embodiments have been shown and described herein, it will be obvious to those skilled in the art that such embodiments are provided by way of example only. Numerous variations, changes, and substitutions may occur to those skilled in the art without departing from devices, systems and methods disclosed herein. It should be understood that various alternatives to the embodiments described herein may be employed.

Unless otherwise defined, all technical terms used herein have the same meaning as commonly understood by one of ordinary skill in the art to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

### Sequence Annotation

Knowledge about the capacity of any single sequence to cause some type of harm may be extremely distributed. Individual communities of researchers focus on widely varying aspects of pathogenicity including the ability of organisms to infiltrate host cells, hijack host cellular machinery, hide from the host immune system and even to enhance the host immune response. Exemplary harmful biological sequences include those that encode for a pathogenic sequence, such as those which are harmful and from viral, bacterial, or parasitic origins. Harmful biological sequences may include be mutant form of wildtype sequences which are known to have pathogenic effects. Harmful biological sequences include sequences that produce harmful sequence products after transcription or translation, or act as precursors to harmful sequence products. Harmful biological sequences include sequences that encode for harmful proteins.

Among other facets, the present disclosure provides for a Mediawiki-based user interface that allows a user to submit sequences along with tag-based annotation of roles in pathogenicity. Users may be encouraged to submit several tags for each sequence to describe the general patterns of harm associated with a given sequence modeled as:

Host+Context=Outcome+Level of Concern

The present system may take a tag-based approach so as not a priori to impose a single controlled vocabulary. The collection of tags resulting from community annotation could form the basis of such a controlled vocabulary over the longer term.

As each sequence is uploaded, users may be asked to add tags in each of four categories. Tagging ‘Host’ and ‘Level of Concern’ are mandatory; adding tags for ‘Context’ and ‘Outcome’ are optional given the additional complexity and domain knowledge required.

As an example, a sequence encoding the toxin ricin might be tagged by a user as:

| Tag | Values |
|---|---|
| Host | Human |
| Context | ingestion, inhalation |
| Outcome | fever, cough, respiratory failure, death |
| Level of | Extreme |
| Concern | |

The goal is accumulation of metadata over time more than universal completeness. The system is centrally hosted and offers the entire set of curated sequences (or subsets based on queries by tag) for download as FASTA for use in screening.

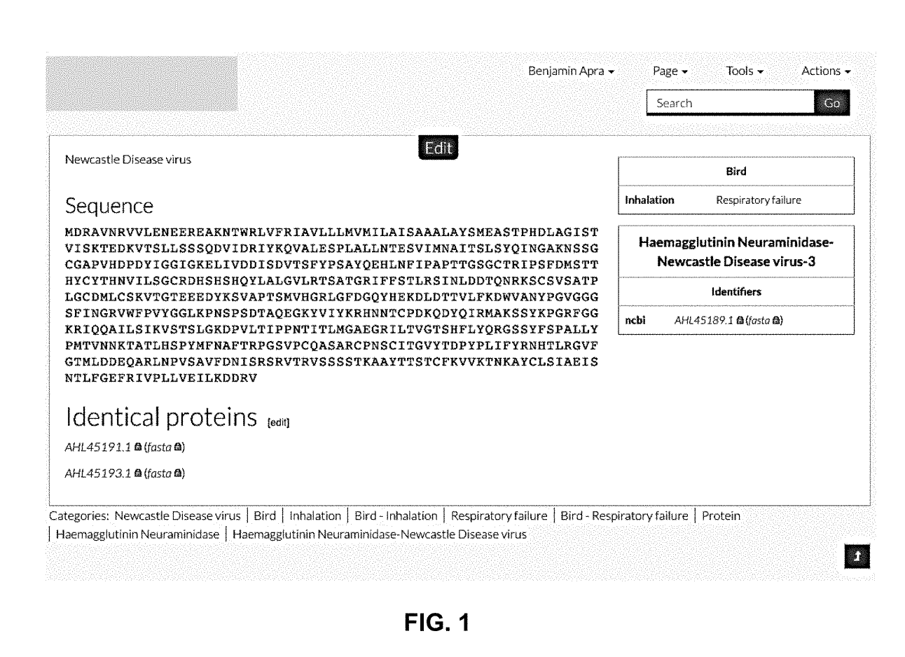

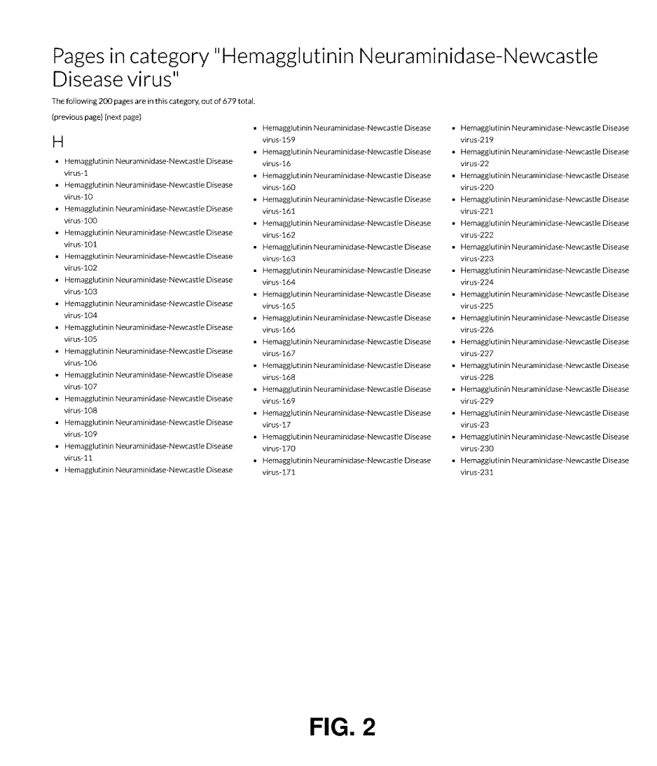

Provided herein are methods for sequence annotation wherein a database receives a listing of characteristics associated with a biological sequence or biological construct (e.g., nucleotide sequence or protein sequence). Exemplary characteristics include, without limitation: nucleic acid sequence, protein sequence, protein name, strain source, link to sequence database (e.g., NCBI), sequence database accession number, identical sequences (protein or nucleic acid), similar sequences (protein or nucleic acid), disease type (e.g., virus, bacterium, or fungi), host information (e.g., humans, mammals, birds, insects), context or route of harmful interaction (e.g., ingestion, inhalation), and level of concern. Also provided herein is a user interface which presents each characteristic or a link to additional information of such characteristics. See FIG. 1. In some cases, viral sequences for a particular strain are selected. For example, FIG. 2 illustrates a portion of 679 available strains of Hemagglutinin Neuraminidase-Newcastle Disease virus for annotation.

Exemplary species include animal species. “Animals” as used herein includes, without limitation, mammals, marsupials, birds, insects, arthropods, amphibians and reptiles. Exemplary mammals include, without limitation, sheep, cattle, goats, pigs, rabbits, hares, deer, goats, mice, rats, bats, and possums, and the like. Exemplary disease types include pathogens from the following classes: viruses, bacterium, fungi and other harmful pathogens. Exemplary viruses having harmful expression products include, without limitation, Marburg virus, Ebola virus, Hantavirus, bird flu (e.g., H5N1 strain), Lassa virus, Junin virus, Crimea-Congo fever, Machupo virus, Kyasanur Forest Virus, Dengue fever, and Chikungunya virus. Exemplary bacterium having harmful expression products include, without limitation, Multi-Resistant Staphylococcus aureus (MRSA), E. coli, listeriosis, salmonella, gonococcus, streptococcus and staphylococcus. Exemplary fungi having harmful expression products include, without limitation, Amanita arocheae, Amanita bisporigera, Amanita exitialis, Amanita magnivelaris, Amanita ocreata, Amanita verna, Clitocybe dealbata, Cortinarius gentilis, Lepiota brunneoincarnata, Lepiota brunneoincarnata, Lepiota brunneoincarnata, and Lepiota brunneoincarnata. Exemplary routes to harm include, without limitation, ingestion, inhalation, skin contact, and sexual transmission. Exemplary outcomes include, without limitation, fever, headache, nausea, dizziness, and diarrhea. Exemplary protein databases include US National Library of Medicine National Institutes of Health protein and gene databases. Exemplary levels of disease concern include low, medium, high, and extreme.

Provided herein are methods for basic curation, such as identifying a sequence associated with a query by organism name and or taxon. Once identified, a sequence annotation may optionally be updated and, optionally, recategorized for a particular descriptive feature. Sequences identified are further available for downloading in a singular or batch format, optionally with FASTA formatting.

Data quality and public participation can both be concerns associated with publicly available databases. To maximize immediate utility, the disclosed system may carry out an initial curation process adding many pathogenic proteins to the database in an attempt to include most potentially regulated sequences or other sequences known to be harmful. The system may curate an “unrestricted” list of NCBI GI identifiers corresponding to genes that may be considered harmless. That unrestricted list may be also open to curation.

A scheme of CAPTCHA may be used to prevent bot-driven curation and require user registration before creating or editing pages. GI identifiers may be periodically verified (for existence), and records may be tagged for human review on failure. Users can also flag records to request community or administrator review.

The present disclosure provides for systems and methods that annotate and/or screen at least one biological sequence. In some instances, the biological sequence is a nucleic acid sequence. The nucleic acid sequence may comprise 1; 10; 100; 200; 300; 400; 500; 600; 700; 800; 900; 1,000; 2,000; 5,000; 7,000; 10,000, or more nucleic acid residues. In some instances, the nucleic acid sequence comprises between 100 and 500 nucleic acid residues. In some instances, the nucleic acid sequence comprises between 50 and 1000 nucleic acid residues. In some instances, the nucleic acid sequence comprises between 20 and 200 nucleic acid residues. In some instances, the nucleic acid sequence comprises 200 residues. In some instances, the biological sequence may be DNA or RNA. In some instances, the biological sequence is a protein sequence. The biological sequence may comprise adenine (A), cytosine (C), guanine (G), thymine (T), or uracil (U). In some instances, the biological sequence is a protein sequence. The protein may comprise 1; 10; 100; 200; 300; 400; 500; 600; 700; 800; 900; 1,000; 2,000 or more amino acids. In some instances, the protein sequence comprises between 100 and 300 amino acids. In some instances, the nucleic acid sequence comprises between 50 and 500 amino acids. In some instances, the nucleic acid sequence comprises between 10 and 200 amino acids. In some instances, the nucleic acid sequence comprises 60 amino acids. In some instances, nucleic acid fragments of no more than 2, 5, 10, 20, 50, 100, or 200 residues are assembled in-silico into a nucleic acid sequence. In some instances, nucleic acid fragments are obtained from one or more sources, or one or more orders from the same source.

### Screening Tool

Constructing a screening system capable of determining whether a given sequence poses a biosecurity risk may include a degree of investment in time and expertise not available to all synthetic biologists or even to all synthetic biology companies. Even assuming one has access to a database of dangerous sequences, basic parameterization of an aligner and result processing (including culling alignment counts to similar regions so as not to hide homology to shorter regions) may include domain expertise.

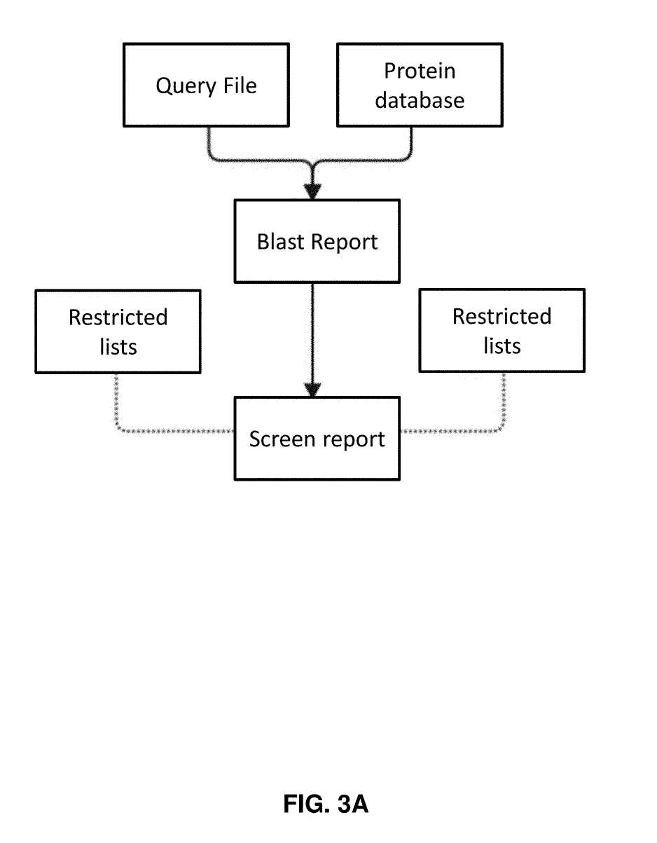

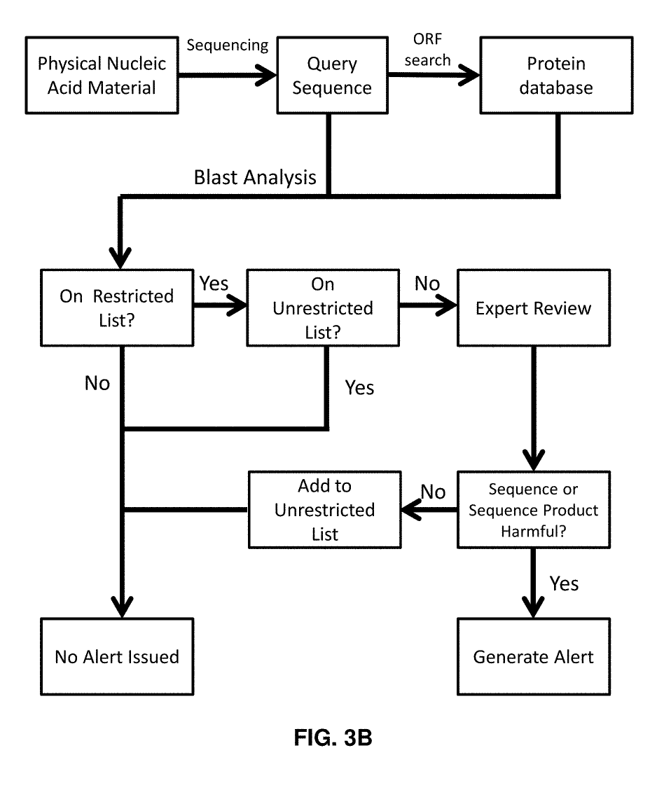

An illustrative workflow is provided in FIG. 3A. Referring to FIG. 3A, processor receives a query file containing biological sequence information, and is also in communication with a protein database having identified sequence information. A BLAST report is generated listing the same and similar sequences identified associated with the queried biological sequence, in-part or whole. The BLAST report is then queried to databases containing sequence annotations identifying sequences associated with harmful biological sequences (protein or nucleic acids), also referred to as “restricted” lists. A screen report is generated in the form of a user interface which summarizes the results of these processes.

An illustrative logic workflow is provided in FIG. 3B. Referring to FIG. 3B, a data input source such as physical nucleic acid or protein material (which can be sequenced), a nucleic acid sequence (which can be translated into a protein sequence), or a protein sequence can be evaluated using an algorithm which searches one or more databases to determine if it is on a restricted list. Exemplary algorithms include but are not limited to, BLAST, DIAMOND, Smith-Waterman, or other algorithm for comparing sequence information. Sequences found to be on the restrictive list are further evaluated against an unrestricted list that comprises known false positives. If no false positive is identified, the sequence is subjected to expert review. If the sequence is found to be non-harmful, it is placed on the unrestricted list to prevent further identification of said sequence as a false positive. If the sequence is found to be harmful, an output alert is generated. In some instances, the non-harmful sequence is synthesized. In some instances, the sequence is modified to remove the harmful sequence. In some instances, the modified sequence is re-screened. In some instances, this process is repeated iteratively until a modified non-harmful sequence is found. In some instances, the modified non-harmful sequence is synthesized.

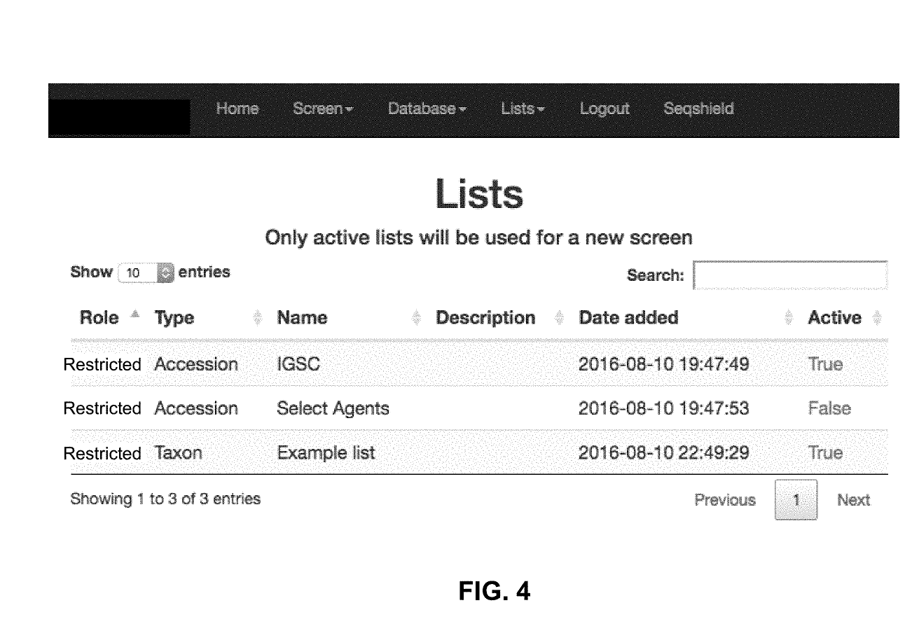

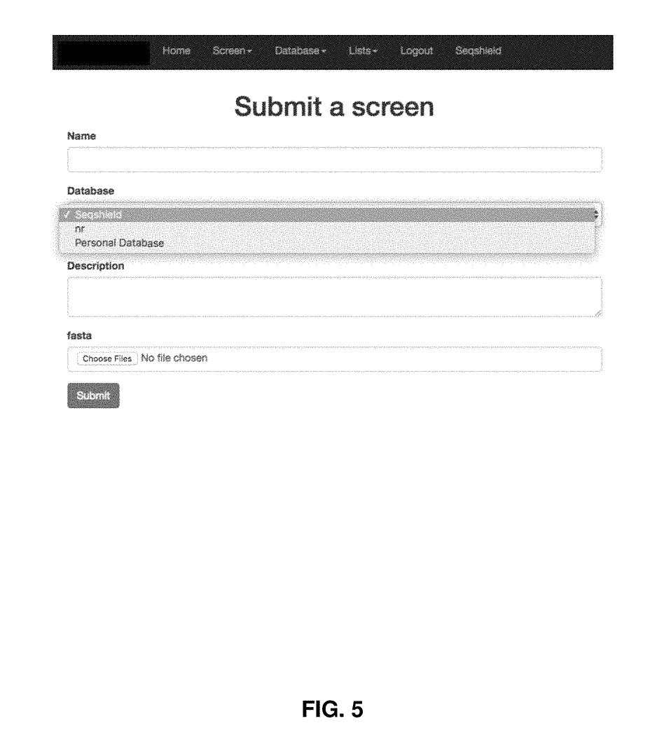

Referring to FIG. 4, a user interface displays restricted lists available for selection for the screening process. Referring to FIG. 5, an illustrative user interface displays a “Submit a screen” submission form. The form allows for selection of screening against open database(s), e.g., a collection of publically available information, or screening against a personal database, which may be based on a non-publicly available selection criteria. The submission form also allows for selection of a biological sequence file for uploading.

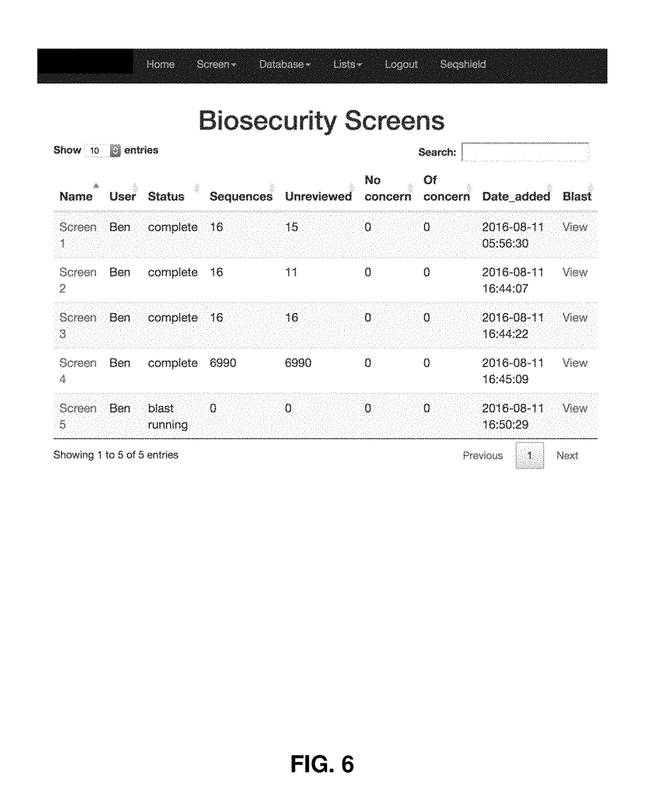

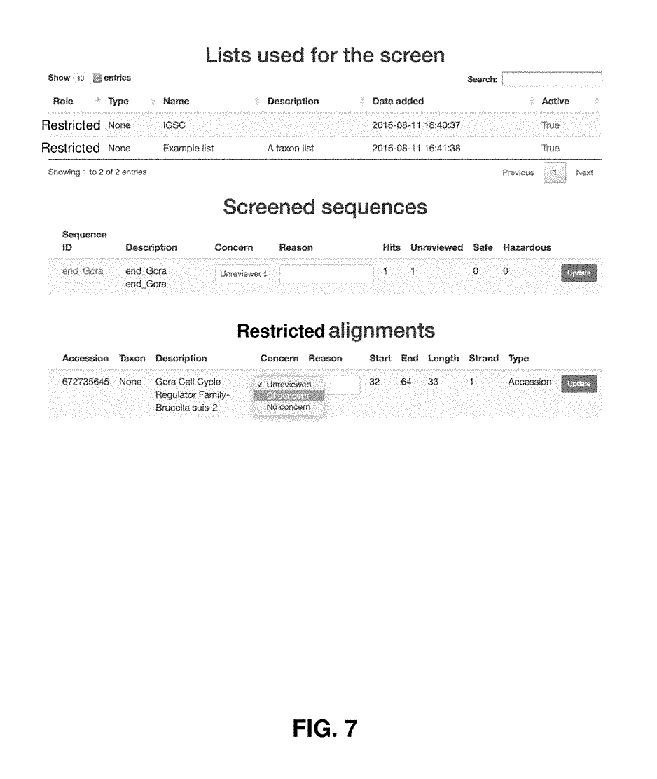

Referring to FIG. 6, an illustrative user interface displays a summary of Biosecurity screens conducted, with status information, sequences screened, review status, concern or no concern status, date of sequence addition, and a link to viewing the BLAST result. Referring to FIG. 7, an illustrative user interface displays a summary of lists accessed during a screen, sequences screened, and harmful sequence (restricted) assignments for a sequence.

The technologies disclosed herein may comprise a Python-based reference implementation of a screening system. Given a query nucleotide sequence, the system may compare the sequence (e.g., via BLAST) to the set of protein sequences derived from the annotated collection produced by the interface discussed in the previous section.

Results may be filtered by the degree of homology, E-score and alignment length. Passing hits may be summarized by the distribution of tags associated with those sequences and the regions of the query found problematic. Links to the originating database entries may be provided so that users can follow-up in more detail. In compliance with pre-defined guidance, some examples show that the algorithm is 100% sensitive and reports can be downloaded for archival use. Screening short (e.g., less than about 200 bases) sequences may result in a large number of false positive findings. Effective screening of shorter polynucleotide sequences may include an algorithmic approach.

The screening system may sit atop a database and include a RESTful application programmable interface (API) for screen request submission and result retrieval as well as a graphical user interface. The application may be installed and operate on a laptop computer, and scale reasonably well to high-throughput use via API calls.

### Cumulative Biological Sequence or Construct Screening

It is possible to obtain fragments of biological sequences or constructs that when individually screened will not result identification of a harmful sequence, especially if the biological sequences or constructs are obtained through multiple sources and at multiple time points. In some instances, the source may be a customer. For example, accumulation of a substantial portion of the genome of any of the select agent-regulated bacteria or viruses may be obtained in smaller pieces, and then assembled into a harmful biological sequence or construct. To address this, in some instances a background process after each request is received which queries a database for all previous orders from that biological sequence or construct requesting source and collects records of any segments with high homology to any harmful biological sequences or constructs. This ensures evaluation and alerting even if those segments were insufficient to trigger formal alerting or denial of possession during the individual order. In some instances, these high-homology segments are represented as intervals on the genome of the select agent of concern and then the union of all intervals, per a biological sequence or construct requesting source and per genome, is generated to determine a maximum theoretical construction of these organisms per biological sequence or construct requesting source. In some instances, once any biological sequence or construct requesting source seeks to design 20% or more of a given select agent genome, an alert is generated for human review and follow up with the biological sequence or construct requesting source on intent. In some instances, once any biological sequence or construct requesting source can generate at least 5%, 10%, 20%, 30%, 40%, 50%, 60%, 70%, 80%, 90%, or more than 90% of a harmful biological sequence or construct, an alert is generated for human review prior to authorizing sequence building. In some instances, once any biological sequence or construct requesting source can generate between 5% and 50%, betweem 10% and 75%, between 20% and 90%, between 30% and 100%, between 10% and 30%, between 5% and 50%, or between 15% and 60% of a harmful biological sequence or construct, an alert is generated for human review prior to authorizing sequence building.

In some instances, a biological sequence comprises nucleic acids or a protein. For shorter nucleic acid sequences, such as those comprising no more than 200 bases, existing screening methods have very high false positive rates. In some instances a shorter nucleic acid sequence contains no more than 2000, 1000, 500, 200, 100, 75, 50, 40, 30, or no more than 20 bases. In some instances a shorter nucleic acid sequence contains between 10 and 1000 bases, between 20 and 500 bases, between 30 and 300 bases, between 40 and 200 bases, between 50 and 200 bases, between 20 and 200 bases, between 10 and 100 bases, or between 100 and 300 bases. In some instances nucleic acid sequences encode for a shorter protein that comprises no more than 300, 200, 100, 75, 50, 40, 30, 20, 10, 5, or no more than 5 amino acids. In some instances a shorter nucleic acid sequence contains between 10 and 300 amino acids, between 20 and 200 amino acids, between 30 and 100 amino acids, between 10 and 200 amino acids, between 20 and 100 amino acids, between 5 and 50 amino acids, between 10 and 100 amino acids, or between 25 and 75 amino acids. In one example, an alternative screening approach is employed that looks across sets of polynucleotides to determine when a biological sequence or construct requesting source has submitted a request for enough polynucleotides to potentially assemble a regulated or harmful biological sequence or construct. In some instances during ordering, a background process, within one or more sources, assembles polynucleotides across orders against the genomes of select harmful organisms using assembly algorithms. In some instances, assembly algorithms comprise next generation sequencing assembly algorithms. These assemblies allow for hypothesis generation that connect one or more orders with one or more sources. For example, orders X, Y and Z from sources A and B are combined to assemble one or more genes from a harmful organism. In some instances, the number of sources is at least 2, 3, 4, 5, 8, 10, 15, 20, 30, or more than 30 sources. In some instances, the number of sources is between 2 and 30 sources, between 5 and 50 sources, between 10 and 100 sources, between 5 and 20 sources, between 2 and 10 sources, between 4 and 40 sources, or between 15 and 75 sources. In some instances, the hypotheses generate alerts for human review and optionally triggers follow-on discussion with the biological sequence or construct requesting source or reports to law enforcement directly. False positive rates should remain low given the low probability of high homology to gene-length sequences. In some instances, additional false positive reduction comes in the form of evaluating the alignment structure of the hypothesized collection of sequences to determine if proper overlaps would allow assembly of one or more harmful biological sequences or constructs.

In some instances, a physical nucleic acid sample such as a vector or insert is provided by a source for assembly with one or more nucleic acid sequences to be synthesized. In some instances, these physical nucleic acid materials are first sequenced, such as with NGS, and the hypothetical assembly of one or more vector and insert sequences is subjected to screening. In some instances, the combination of at least two sequences is screened. In some instances, the combination of at least 2, 3, 4, 5, 10, 15, 20, 30, or more than 30 sequences is screened for harmful biological sequences or constructs. In some instances, the number of sequences screened is between 2 and 30 sequences, between 5 and 50 sequences, between 10 and 100 sequences, between 5 and 20 sequences, between 2 and 10 sequences, between 4 and 40 sequences, or between 15 and 75 sequences is screened for harmful biological sequences or constructs.

### Digital Processing Device

In some examples, the platforms, systems, media, and methods described herein may include a digital processing device, or use of the same. In some examples, the digital processing device may include one or more hardware central processing units (CPUs) or general purpose graphics processing units (GPGPUs) that carry out the device's functions. In some examples, the digital processing device may further comprise an operating system configured to perform executable instructions. The digital processing device may be optionally connected a computer network. The digital processing device may be optionally connected to the Internet such that it accesses the World Wide Web. The digital processing device may be optionally connected to a cloud computing infrastructure. The digital processing device may be optionally connected to an intranet. The digital processing device may be optionally connected to a data storage device.

In accordance with the description herein, suitable digital processing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Many smartphones may be suitable for use in the system described herein. Televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some examples, the operating system may be provided by cloud computing. The device may include a storage and/or memory device. The storage and/or memory device may be one or more physical apparatuses used to store data or programs on a temporary or permanent basis. The device may be volatile memory and may require power to maintain stored information. The device may be non-volatile memory and retains stored information when the digital processing device is not powered. The non-volatile memory may comprise flash memory, dynamic random-access memory (DRAM), ferroelectric random access memory (FRAM), phase-change random access memory (PRAM).

The digital processing device may include a display to send visual information to a user. The display may be a cathode ray tube (CRT), a liquid crystal display (LCD), a thin film transistor liquid crystal display (TFT-LCD), an organic light emitting diode (OLED) display, a passive-matrix OLED (PMOLED) or active-matrix OLED (AMOLED) display, a plasma display, and/or a video projector.

The digital processing device may include an input device to receive information from a user. The input device may be a keyboard. The input device may be a pointing device including, by way of non-limiting examples, a mouse, trackball, track pad, joystick, game controller, or stylus. The input device may be a touch screen or a multi-touch screen. The input device may be a microphone to capture voice or other sound input. The input device may be a video camera or other sensor to capture motion or visual input. The input device may be a Kinect, Leap Motion, or the like. The input device may be a combination of devices such as those disclosed herein.

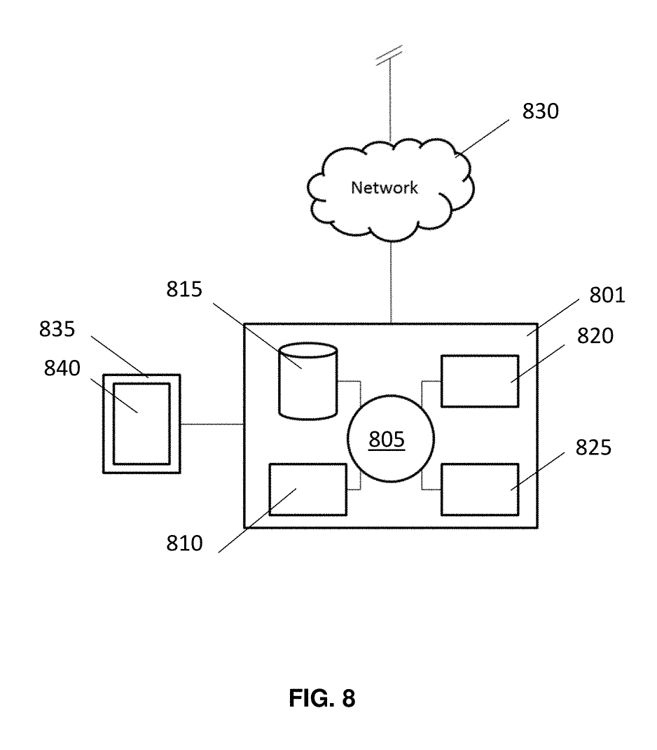

Referring to FIG. 8, in a particular embodiment, an exemplary digital processing device 801 is programmed or otherwise configured to perform annotation or screening. In this example, the digital processing device 801 includes a central processing unit (CPU, also “processor” and “computer processor” herein) 805, which can be a single core or multi core processor, or a plurality of processors for parallel processing. The digital processing device 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The digital processing device 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the device 801, can implement a peer-to-peer network, which may enable devices coupled to the device 801 to behave as a client or a server.

Continuing to refer to FIG. 8, the CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and write back. The CPU 805 can be part of a circuit, such as an integrated circuit. One or more other components of the device 801 can be included in the circuit. In some cases, the circuit is an application specific integrated circuit (ASIC) or a field programmable gate array (FPGA).

Continuing to refer to FIG. 8, the storage unit 815 can store files, such as drivers, libraries and saved programs. The storage unit 815 can store user data, e.g., user preferences and user programs. The digital processing device 801 in some cases can include one or more additional data storage units that are external, such as located on a remote server that is in communication through an intranet or the Internet.

Continuing to refer to FIG. 8, the digital processing device 801 can communicate with one or more remote computer systems through the network 830. For instance, the device 801 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

### Additional Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

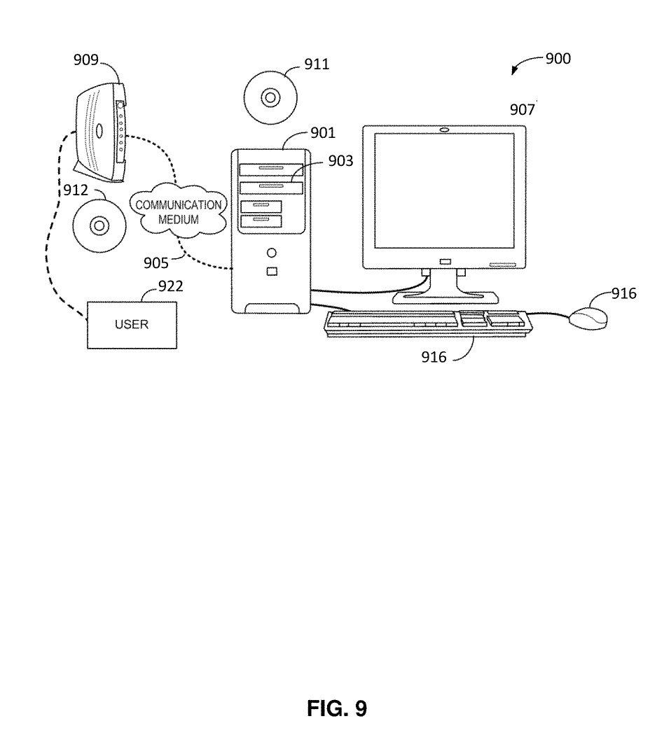

The computer system 900 illustrated in FIG. 9 may be understood as a logical apparatus that can read instructions from media 911 and/or a network port 905, which can optionally be connected to server 909 having fixed media 912. The system, such as shown in FIG. 9 can include a CPU 901, disk drives 903, optional input devices such as keyboard 915 and/or mouse 916 and optional monitor 907. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 922 as illustrated in FIG. 9.

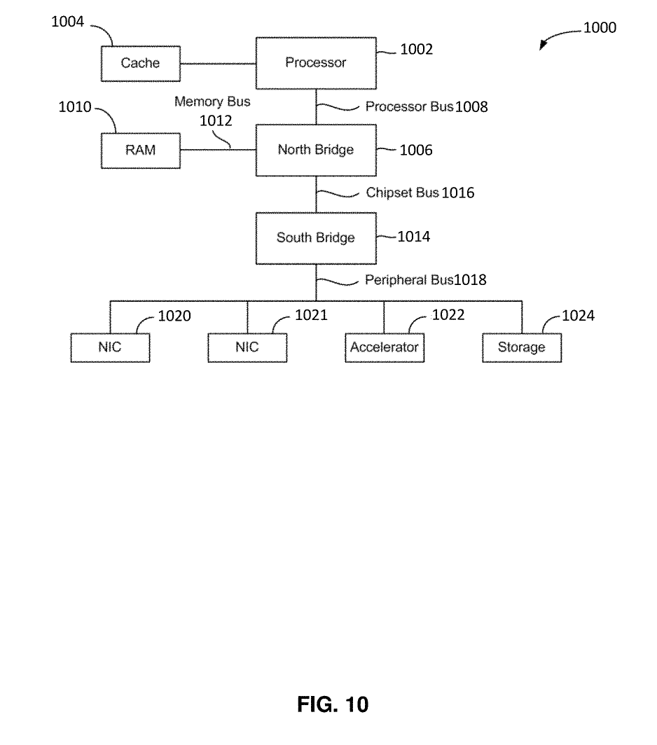

FIG. 10 is a block diagram illustrating a first example architecture of a computer system 1000 that can be used in connection with example instances of the present disclosure. As depicted in FIG. 10, the example computer system can include a processor 1002 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 10, a high speed cache 1004 can be connected to, or incorporated in, the processor 1002 to provide a high speed memory for instructions or data that have been recently, or are frequently, used by processor 1002. The processor 1002 is connected to a north bridge 1006 by a processor bus 1008. The north bridge 1006 is connected to random access memory (RAM) 1010 by a memory bus 1012 and manages access to the RAM 1010 by the processor 1002. The north bridge 1006 is also connected to a south bridge 1014 by a chipset bus 1016. The south bridge 1014 is, in turn, connected to a peripheral bus 1018. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1018. In some alternative architectures, the functionality of the north bridge can be incorporated into the processor instead of using a separate north bridge chip. In some instances, system 1000 can include an accelerator card 1022 attached to the peripheral bus 1018. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1024 and can be loaded into RAM 1010 and/or cache 1004 for use by the processor. The system 1000 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 1000 also includes network interface cards (NICs) 1020 and 1021 connected to the peripheral bus for providing network interfaces to external storage, such as Network Attached Storage (NAS) and other computer systems that can be used for distributed parallel processing.

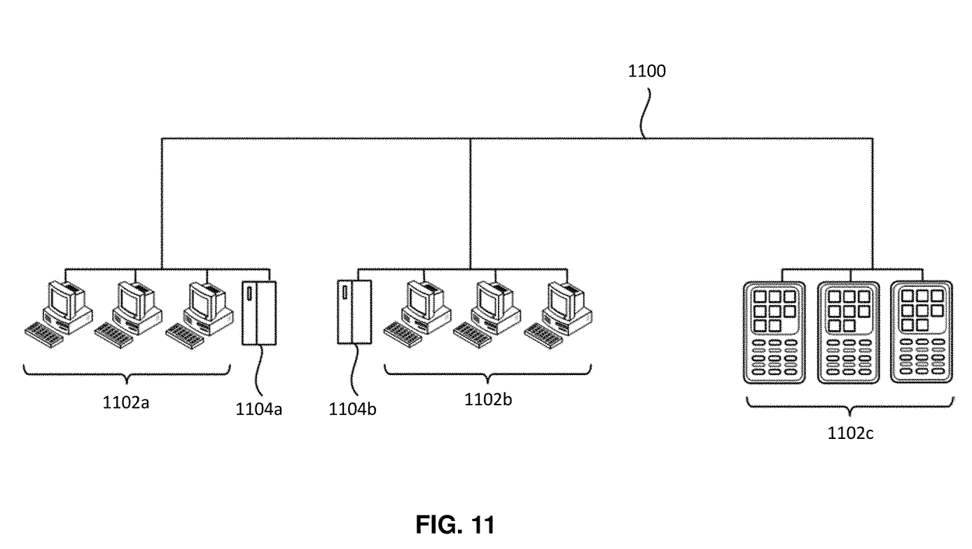

FIG. 11 is a diagram showing a network 1100 with a plurality of computer systems 1102a, and 1102b, a plurality of cell phones and personal data assistants 1102c, and Network Attached Storage (NAS) 1104a, and 1104b. In example instances, systems 1102a, 1102b, and 1102c can manage data storage and optimize data access for data stored in Network Attached Storage (NAS) 1104a and 1104b. A mathematical model can be used for the data and be evaluated using distributed parallel processing across computer systems 1102a, and 1102b, and cell phone and personal data assistant systems 1102c. Computer systems 1102a, and 1102b, and cell phone and personal data assistant systems 1102c can also provide parallel processing for adaptive data restructuring of the data stored in Network Attached Storage (NAS) 1104a and 1104b. FIG. 11 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

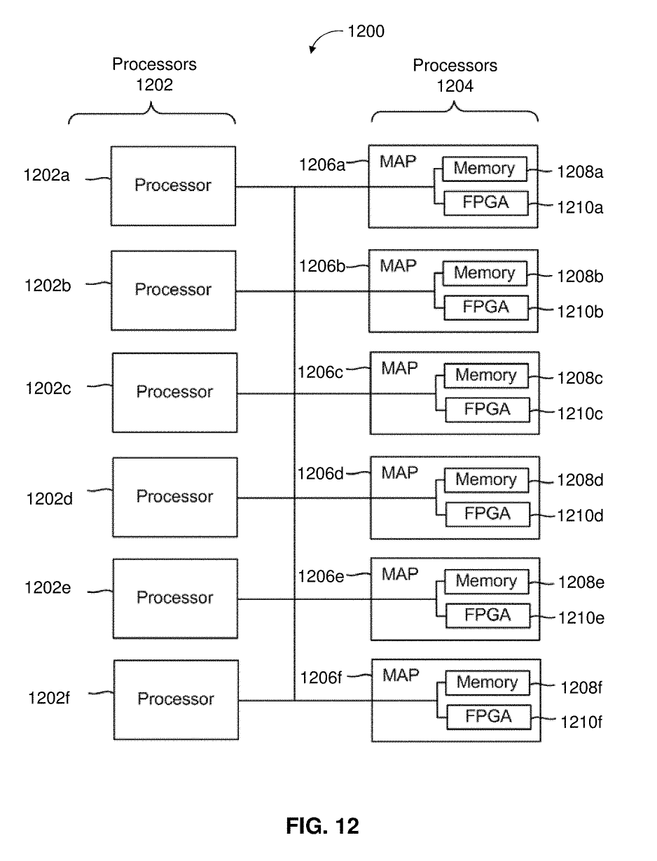

FIG. 12 is a block diagram of a multiprocessor computer system 1200 using a shared virtual address memory space in accordance with an example instance. The system includes a plurality of processors 1202a-f that can access a shared memory subsystem 1204. The system incorporates a plurality of programmable hardware memory algorithm processors (MAPs) 1206a-f in the memory subsystem 1204. Each MAP 1206a-f can comprise a memory 1208a-f and one or more field programmable gate arrays (FPGAs) 1210a-f. The MAP provides a configurable functional unit and particular algorithms or portions of algorithms can be provided to the FPGAs 1210a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1208a-f, allowing it to execute tasks independently of, and asynchronously from the respective microprocessor 1202a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 12, system on chips (SOCs), application specific integrated circuits (ASICs), or other processing and logic elements. For example, the Set Processor and Optimizer can be implemented with hardware acceleration through the use of a hardware accelerator card, such as accelerator card 1022 illustrated in FIG. 10.

### Non-Transitory Computer Readable Storage Medium

The platforms, systems, media, and methods disclosed herein may include one or more non-transitory computer readable storage media encoded with a program including instructions executable by the operating system of an optionally networked digital processing device. A computer readable storage medium may be a tangible component of a digital processing device. A computer readable storage medium is optionally removable from a digital processing device. A computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein may include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, a computer program may be written in various versions of various languages.

### Web Application

A computer program may include a web application. In light of the disclosure provided herein, a web application may utilize one or more software frameworks and one or more database systems. A web application may be created upon a software framework such as Microsoft® .NET or Ruby on Rails (RoR). A web application may utilize one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). A web application may be written to some extent in a presentation definition language such as Cascading Style Sheets (CSS). A web application may be written to some extent in a client-side scripting language such as Asynchronous Javascript and XML (AJAX), Flash® Actionscript, Javascript, or Silverlight®. A web application may be written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. A web application may be written to some extent in a database query language such as Structured Query Language (SQL).

### Mobile Application

A computer program may include a mobile application provided to a mobile digital processing device. The mobile application may be provided to a mobile digital processing device at the time it is manufactured. The mobile application may be provided to a mobile digital processing device via the computer network described herein.

A mobile application may be created, for example, using hardware, languages, and development environments. Mobile applications may be written in various programming languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

### Standalone Application

A computer program may include a standalone application, which is a program that is run as an independent computer process, not an add-on to an existing process, e.g., not a plug-in. Standalone applications may be compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program.

### Web Browser Plug-in

The computer program may include a web browser plug-in. In computing, a plug-in may be one or more software components that add specific functionality to a larger software application. Makers of software applications support plug-ins to enable third-party developers to create abilities which extend an application, to support easily adding new features, and to reduce the size of an application. When supported, plug-ins may enable customizing the functionality of a software application. For example, plug-ins are commonly used in web browsers to play video, generate interactivity, scan for viruses, and display particular file types. Web browser plug-ins include, without limitation, Adobe® Flash® Player, Microsoft® Silverlight®, and Apple® QuickTime®. The toolbar may comprise one or more web browser extensions, add-ins, or add-ons. In some embodiments, the toolbar comprises one or more explorer bars, tool bands, or desk bands.

Several plug-in frameworks may be available that may enable development of plug-ins in various programming languages, including, by way of non-limiting examples, C++, Delphi, Java™, PHP, Python™, and VB .NET, or combinations thereof.

Web browsers (also called Internet browsers) are software applications, which may be configured for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) may be configured for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry Browser, Apple® Safari®, Palm Blazer, Palm WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The systems, media, networks and methods described herein may include software, server, and/or database modules, or use of the same. Software modules may be created using various machines, software, and programming languages. The software modules disclosed herein are implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. The one or more software modules may comprise, by way of non-limiting examples, a web application, a mobile application, and a standalone application. In some embodiments, software modules are in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The platforms, systems, media, and methods disclosed herein may include one or more databases, or use of the same. In view of the disclosure provided herein, many databases are suitable for storage and retrieval of physiological data. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. A database may be web-based. A database may be cloud computing-based. A database may be based on one or more local computer storage devices.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Algorithms

The platforms, systems, media, and methods disclosed herein may include one or more algorithms, or use of the same. In view of the disclosure provided herein, many algorithms are suitable for searching and comparing sequence data. In various embodiments, suitable algorithms include, by way of non-limiting examples BLAST, DIAMOND, BLAT, BWT, PLAST, Smith-Waterman, or other algorithm for sequence searching and alignment. Algorithms may include accelerated or extended versions of existing algorithms, or software tools which use these algorithms. In some instances, suitable accelerated or extended algorithms and software tools by way of non-limiting examples include CS-BLAST, Tera-BLAST, GPU-Blast, G-BLASTN, MPIBLAST, Paracel BLAST, CaBLAST, or any other additional algorithms or software tools that accelerate the BLAST algorithm.

Provided herein are systems and methods for designing and synthesizing biological sequences or constructs with enhanced biosafety and biosecurity. In some instances, biosafety refers to enhanced safety of individuals, for example, through preventative measures aimed to prevent contact with harmful biological agents during or resulting from manufacture. In some instances, biosecurity refers to protecting the safety of populations, for example, through preventative measures aimed to prevent the use or spread of harmful biological agents. In some instances, one or more biological constructs comprising one or more biological sequences is received, screened for biosecurity risk using a database, and an alert generated if one or more of the biological sequences or constructs is determined to be a harmful expression construct or harmful product. In some instances, biological sequences or constructs refer to synthetic sequences. In some instances, biological sequences or constructs refer to naturally occurring sequences. In some instances, biological sequences or constructs comprise nucleic acids or amino acids. In some instances, biological sequences refer to synthetic sequences. In some instances, biological sequences refer to naturally occurring sequences. In some instances, biological sequences comprise nucleic acids or amino acids. In some instances, user annotation is used to provide additional information concerning properties of biological sequences or constructs in the database. In some instances, the methods and systems are amenable to automation so as to fit seamlessly into high-throughput design/build/test workflows. In some instances, screening a biological construct comprises comparing the combination of smaller biological sequences obtained from single or multiple sources over multiple time points. In some instances, biological sequences or constructs determined to be harmful are further evaluated by a human expert to reduce future false positives. In some instances, these systems and methods comprise computers, software applications, and networks to interface with users and databases.

Provided herein are systems comprising: a processor and a memory; machine instructions for evaluating biosecurity of a biological construct, the machine instructions comprising: a database of a plurality of tags associated with the biological construct; an annotation tool; and, optionally, a screening tool. Further provided herein are systems wherein the biological sequence or construct comprises one or more biological sequences. Further provided herein are systems wherein the biological sequence is a nucleic acid sequence. Further provided herein are systems wherein the biological sequence is a protein sequence. Further provided herein are systems wherein the annotation tool is configured to allow a user to provide one or more annotated tags of a sequence of the biological construct. Further provided herein are systems wherein the one or more annotated tags comprise at least a host and a level of concern. Further provided herein are systems wherein the one or more annotated tags comprise an outcome. Further provided herein are systems wherein the outcome comprises a disease. Further provided herein are systems wherein the one or more annotated tags comprise context. Further provided herein are systems wherein the one or more annotated tags comprise pathogenicity. Further provided herein are systems wherein the one or more annotated tags comprise harm. Further provided herein are systems wherein the one or more annotated tags is based on one or more terms. Further provided herein are systems wherein the one or more annotated tags is based on one or more sentence descriptions. Further provided herein are systems wherein the annotation tool is further configured to generate a controlled vocabulary of the one or more annotated tags. Further provided herein are systems wherein the annotation tool comprises a curation process. Further provided herein are systems wherein the curation process comprises integrating information of the biological sequence or construct from an external database to the database. Further provided herein are systems wherein the curation process comprises determining a harmless feature of the biological construct. Further provided herein are systems wherein the annotation tool comprises aligning the sequence with sequences of the biological sequence or construct in the database. Further provided herein are systems wherein the screening tool is configured to allow a user to search a biosecurity risk of a given sequence of the biological construct. Further provided herein are systems wherein the given sequence comprises a nucleotide sequence. Further provided herein are systems wherein the given sequence comprises a protein sequence. Further provided herein are systems wherein the screening tool comprises a sequence aligner to align the given sequence with sequences of the biological sequence or construct in the database. Further provided herein are systems wherein the searching the biosecurity risk comprises filtering by a degree of homology. Further provided herein are systems wherein the searching the bio security risk comprises evaluating a sequence alignment length. Further provided herein are systems wherein the searching the biosecurity risk comprises generating an evaluation score. Further provided herein are systems wherein the screening tool further comprises an application programmable interface. Further provided herein are systems wherein the machine instructions further comprises a graphical user interface for annotation and screening.

Provided herein are computer-implemented methods for evaluating bio security risk comprising: using, by a processor, a database to store a plurality of tags associated with a biological construct; using, by a processor, an annotation tool to annotate features of the biological construct; and, optionally, using, by a processor, a screening tool to search features of the biological construct. Further provided herein are methods wherein the biological construct comprises a biological sequence. Further provided herein are methods wherein the biological sequence is a nucleic acid sequence. Further provided herein are methods wherein the biological sequence is a protein sequence. Further provided herein are methods wherein the annotation tool is configured to allow a user to provide one or more annotated tags of a sequence of the biological construct. Further provided herein are methods wherein the one or more annotated tags comprise at least a host and a level of concern. Further provided herein are methods wherein the one or more annotated tags comprise an outcome. Further provided herein are methods wherein the outcome comprises a disease. Further provided herein are methods wherein the one or more annotated tags comprise context. Further provided herein are methods wherein the one or more annotated tags comprise pathogenicity. Further provided herein are methods wherein the one or more annotated tags comprise harm. Further provided herein are methods wherein the one or more annotated tags is based on one or more terms. Further provided herein are methods wherein the one or more annotated tags is based on one or more sentence descriptions. Further provided herein are methods wherein the annotation tool is further configured to generate a controlled vocabulary of the one or more annotated tags. Further provided herein are methods wherein the annotation tool comprises a curation process. Further provided herein are methods wherein the curation process comprises integrating information of the biological sequence or construct from an external database to the database. Further provided herein are methods wherein the curation process comprises determining a harmless feature of the biological construct. Further provided herein are methods wherein the annotation tool comprises aligning the sequence with sequences of the biological construct in the database. Further provided herein are methods wherein the screening tool is configured to allow a user to search a biosecurity risk of a given sequence of the biological construct. Further provided herein are methods wherein the given sequence comprises a nucleotide sequence. Further provided herein are methods wherein the given sequence comprises a protein sequence. Further provided herein are methods wherein the screening tool comprises a sequence aligner to align the given sequence with sequences of the biological construct in the database. Further provided herein are methods wherein the searching the biosecurity risk comprises filtering by a degree of homology. Further provided herein are methods wherein the searching the biosecurity risk comprises evaluating a sequence alignment length. Further provided herein are methods wherein the searching the biosecurity risk comprises generating an evaluation score. Further provided herein are methods wherein the screening tool further comprises an application programmable interface. Further provided herein are methods wherein the machine instructions further comprises a graphical user interface for annotation and screening.

Provided herein, are computer-implemented methods for evaluating biosecurity risk, comprising: accessing, by a processor, a database to store a plurality of tags associated with a biological construct; assessing, by a processor, a screening tool to search features of the biological construct; and transmitting, by a processor, a reporting tool to send search results of the screening tool. Further provided herein are methods wherein the biological construct comprises a biological sequence. Further provided herein are methods wherein the biological sequence is a nucleic acid sequence. Further provided herein are methods wherein the biological sequence is a protein sequence. Further provided herein are methods further comprising an annotation tool configured to allow a user to provide one or more annotated tags of a sequence of the biological construct. Further provided herein are methods wherein the one or more annotated tags comprise at least a host and a level of concern. Further provided herein are methods wherein the one or more annotated tags comprise an outcome. Further provided herein are methods wherein the outcome comprises a disease. Further provided herein are methods wherein the one or more annotated tags comprise context. Further provided herein are methods wherein the one or more annotated tags comprise pathogenicity. Further provided herein are methods wherein the one or more annotated tags comprise degree of harm. Further provided herein are methods wherein the one or more annotated tags is based on one or more terms. Further provided herein are methods wherein the one or more annotated tags is based on one or more sentence descriptions. Further provided herein are methods wherein the annotation tool is further configured to generate a controlled vocabulary of the one or more annotated tags. Further provided herein are methods wherein the annotation tool comprises a curation process. Further provided herein are methods wherein the curation process comprises integrating information of the biological sequence or construct from an external database to the database. Further provided herein are methods wherein the curation process comprises determining a harmless feature of the biological construct. Further provided herein are methods wherein the annotation tool comprises aligning the sequence with sequences of the biological construct in the database. Further provided herein are methods wherein the screening tool is configured to allow a user to search a bio security risk of a given sequence of the biological construct. Further provided herein are methods wherein the given sequence comprises a nucleotide sequence. Further provided herein are methods wherein the given sequence comprises a protein sequence. Further provided herein are methods wherein the screening tool comprises a sequence aligner to align the given sequence with sequences of the biological construct in the database. Further provided herein are methods wherein the searching the biosecurity risk comprises filtering by a degree of homology. Further provided herein are methods wherein the searching the biosecurity risk comprises evaluating a sequence alignment length. Further provided herein are methods wherein the searching the biosecurity risk comprises generating an evaluation score. Further provided herein are methods wherein the screening tool further comprises an application programmable interface. Further provided herein are methods further comprising transmitting machine instructions for a graphical user interface for annotation. Further provided herein are methods wherein further comprising transmitting machine instructions for a graphical user interface for screening. Further provided herein are methods further comprising transmitting machine instructions for a graphical user interface for reporting. Further provided herein are methods wherein the biological construct comprises a biological sequence associated with a harmful expression product (e.g., protein resulting from translation) or a harmful product (e.g., RNA resulting from transcription). Further provided herein are methods wherein the biological sequence is viral, bacterial or fungal. Further provided herein are methods further comprising received machine instructions to access the database to store the plurality of tags associated with the biological construct. Further provided herein are methods wherein the machine instructions include information associated with the biological construct. Further provided herein are methods wherein the information associated with the biological sequence or construct comprises a nucleic acid sequence or a protein sequence. Further provided herein are methods wherein the information associated with the biological sequence or construct comprises a database accession number.

It shall be understood that different aspects of the present disclosure can be appreciated individually, collectively, or in combination with each other. Various aspects of the disclosure described herein may be applied to any of the particular applications set forth below. Other objects and features of the present disclosure will become apparent by a review of the specification, claims, and appended figures.

### EXAMPLES

### Example 1: Sequence Annotation

A biological sequence was received by a processor unit. In this example, the biological sequence is a protein sequence. The processor unit accessed a protein database and identified a protein sequence matching the received protein sequence. The processor unit received information associated with various characteristics of the protein sequence. Characteristics included: nucleic acid sequence associated with the protein sequence, the protein sequence, protein name, strain source information, link to sequence database (e.g., NCBI), sequence database accession number, identical sequences (protein or nucleic acid), similar sequences (protein or nucleic acid), disease source (e.g., virus, bacterium), taxonomic description of the organism (e.g., kingdom, phylum, class, order, family, genus, species), host information (e.g., humans, mammals, birds, insects), context or route of harmful interaction (e.g., ingestion, inhalation), a symptom, and level of concern. In this Example, the protein accessed was Newcastle Disease Virus-3. An exemplary user interface provided characteristics for annotating is provided in FIG. 1. When machine instructions were received by the processor with information of characteristics associated with biological sequence, tag information associated with the biological sequence was updated. For example, referring to FIG. 1, Newcastle Disease Virus-3 has tag-information of a protein sequence, identical proteins (AHL4519.1.1 and AHL45193.1), a host type (bird), a route of harmful interaction (inhalation), and a symptom (respiratory failure).

When the processor unit received a selection for the “Hemagglutinin Neuraminidase-Newcastle Disease Virus” family, a listing of virus strain information was accessed and, optionally, transmitted with machine instructions for a user interface to display the strains. See, e.g., FIG. 2, providing a partial listing of 679 available strains of Hemagglutinin Neuraminidase-Newcastle Disease virus for annotation.

Additional tag information consistent with the specification is also used in some instances, including but not limited to FSAP control or Export Control.

### Example 2: Sequence Screening

Referring to FIG. 3A, a processor received machine instructions in the form of query file containing biological sequence information, in this case nucleic acid information. The processor was also in communication with nucleic acid and protein databases. The processor accessed the nucleic acid and protein databases. A BLAST processed report was generated listing the same and similar sequences identified as associated with the queried biological sequence, in-part or whole. Sequences from the BLAST processed report were then queried to databases containing sequence annotations identifying sequences associated with harmful biological sequences (protein or nucleic acids), also referred to as “restricted” lists. A screen report was generated in the form of a user interface which summarizes the results of these processes. The screen report was transmitted in the form of machine instructions for a user interface. The processor received specific instructions for databases to access the restricted list information. See FIG. 4. The restricted lists may be open over the internet or closed and only accessible with authorization. A screen report was also generated to include a summary of biological sequence screens. 5 screens were conducted. See FIG. 6. A screen report was also generated to include a listing of “restricted assignments,” identified harmful biological sequences. See FIG. 7. The screen report identified Gcra Cell Cycle Regulatory Family-Brucella suis-2 protein.

### Example 3: Pre-Screening Against Specific Genomes

Access to more than 500 nucleotides of the genome of Variola major or minor is restricted by World Health Organization (WHO) policy. Those wanting longer sequences must apply for and be granted permission by WHO prior to synthesis. Because of the unique nature of Variola, a pre-screening against just the genomes of Variola major and Variola minor along with Vaccinia and other closely-related Orthopox viruses is conducted. A nucleic acid sequence was evaluated using the general biosecurity screening procedure of Example 2 and the genomes of Orthopox viruses. This screening was carried out in less than 1 second (via blastx on commodity hardware). Vaccinia and other orthopox reference sequences were included to make sure the homology of the requested sequence is greatest to Variola (akin to the 2010 HHS guidance ‘best match’ criteria) prior to alerting.

This could be performed optionally during an order quote-generation process where, if a harmful sequence is detected, an alert is generated for human review prior to starting manufacture.

### Example 4: Library Template Screening

A gene-length nucleic acid sequence of about 600 nucleotides encoding a gene encoding for about 200 amino acids was selected for the production of a variant library. The sequence was obtained and submitted to the general biosecurity screening procedure of Example 2 to ensure that variant library will not contain harmful sequences. The program was designed to generate an alert for human review when a harmful sequence is detected.

### Example 5: Custom Nucleic Acid Screening

A physical nucleic acid-containing material, such as a vector, was obtained and sequenced via Next Generation Sequencing (NGS). The consensus sequence data obtained from NGS was submitted to the general biosecurity screening procedure of Example 2. This ensures that the nucleic acid material does not pose a biosecurity or biosafety concern, such as by encoding for expression of a toxin in a vector backbone away from the insertion site intended for use, such that transformation into E. coli would result in expression of a harmful agent, such as a toxin. The program was designed to generate an alert for human review when a harmful sequence is detected.

### Example 6: Within-Same Query, Cross-Order Assemblies Against Select Agent Genomes

To manage the risk that a requestor (a biological sequence or construct requesting source, such as a customer) may, over time and across individual orders, accumulate a substantial portion of the genome of any of the select agent-regulated bacteria or viruses, a background process after each requestor queries the database for all previous orders from that requestor and collects records of any segments with high homology to any of the select agent bacteria or viruses using the general method of Example 2. This ensures evaluation and alerting even if those regions were insufficient to trigger formal alerting or denial of possession during the individual order. These high-homology segments are represented as intervals on the genome of the select agent of concern and then the union of all intervals, per requestor and per genome, is generated to determine the maximum theoretical construction of these organisms per requestor. Once any requestor can generate 20% or more of a given select agent genome, an alert is generated for human review and follow up with the requestor on intent.

### Example 7: Polynucleotide Pool Assembly Against Select Agent Genomes for Hypothesis Generation

For shorter polynucleotide sequences, such as those containing no more than 200 bases, existing screening methods have very high false positive rates. An alternative screening approach is employed that looks across sets of polynucleotides to determine when a requestor (a biological sequence or construct requesting source, i.e. a customer) has ordered enough polynucleotides to potentially assemble a regulated or harmful sequence. During ordering, a background process, within one or more requesting sources, assembles polynucleotides across orders against the genomes of select agent bacteria and viruses using assembly algorithms from NGS. These assemblies allow for hypothesis generation, such as “If orders X, Y and Z from requestors A and B are combined, three genes from Variola could be fully assembled.” These hypotheses generate alerts for human review and optionally trigger follow-on discussion with requestors or reporting to law enforcement directly. False positive rates should remain low given the low probability of high homology to gene-length sequences; additional false positive reduction comes in the form of evaluating the alignment structure of the hypothesized collection of polynucleotides to determine if proper overlaps that would allow easy assembly exist (i.e. does it appear to have been designed with intent in mind).

### Example 8: Machine Learning-Guided Risk Annotation

A screening platform and human review build a large unrestricted list and a set of true positive alert cases in which a biological sequence or construct requesting source was confirmed as ordering restricted sequences of concern. Machine learning algorithms are trained on both the sequence itself (e.g. Hidden Markov Model (HMM)-type context-aware state models) and/or on the GenBank record annotation (e.g. natural language processing (NLP)-type models to estimate the probability of future unrestricted sequence assignment based on shared language and meaning with previously unrestricted sequence listed records).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. A computerized system for providing enhanced polynucleotide synthesis:
a) a server for hosting a database, wherein the database is adapted for representing a list of harmful biological sequences;
b) a network connection; and
c) a computer readable medium comprising instructions for a general purpose computer, wherein said computerized system is configured for operating in a method of:
i) receiving one or more design instructions, wherein the design instructions comprise a plurality of biological sequences, wherein each of the biological sequences is no more than 500 bases in length, and wherein the plurality of biological sequences comprise a nucleic acid or amino acid sequence;
ii) automatically determining whether at least two of the plurality of biological sequences collectively corresponds to at least 20% of a harmful biological sequence in the database; and
iii) automatically generating an alert if at least 20% of the harmful biological sequence is detected.

2. The system of claim 1, further comprising wherein if no alert is generated, then one or more polynucleotides are synthesized, wherein the one or more polynucleotides encode for the plurality of biological sequences.

3. The system of claim 1, further comprising receiving instructions for changing the at least two of the plurality of biological sequences corresponding to at least 20% of the harmful biological sequence to remove the harmful biological sequence.

4. The system of claim 1, wherein the one or more design instructions are received at one or more time points.

5. The system of claim 1, wherein the one or more design instructions are from different sources.

6. (canceled)

7. (canceled)

8. (canceled)

9. The system of claim 1, wherein the plurality of biological sequences are each no more than 200 bases in length.

10. (canceled)

11. (canceled)

12. The system of claim 9, wherein the plurality of biological sequences are each no more than 20 bases in length.

13. A method for providing enhanced polynucleotide synthesis comprising:
a) receiving one or more design instructions, wherein the design instructions comprise a plurality of biological sequences, wherein each of the biological sequences is no more than 500 bases in length, and wherein the plurality of biological sequences comprise a nucleic acid or amino acid sequence;
b) automatically determining whether at least two of the plurality of biological sequences collectively corresponds to at least 20% of a harmful biological sequence in a database; and
c) automatically generating an alert if at least 20% of the harmful biological sequence is detected.

14. The method of claim 13, further comprising wherein if no alert is generated, then one or more polynucleotides are synthesized, wherein the one or more polynucleotides encode for the plurality of biological sequences.

15. The method of claim 13, further comprising receiving instructions for changing the at least two of the plurality of biological sequences corresponding to at least 20% of the harmful biological sequence to remove the harmful biological sequence.

16. A computerized system for providing enhanced polynucleotide synthesis:
a) a server for hosting a database, wherein the database is adapted for representing a list of sequences;
b) a network connection; and
c) a computer readable medium comprising instructions for a general purpose computer, wherein said computerized system is configured for operating in a method of:
i) receiving one or more design instructions, wherein the design instructions comprise a plurality of biological sequences, wherein the plurality of biological sequences comprises a vector sequence, and a plurality of additional insert sequences;
ii) automatically determining whether the vector and at least one of the plurality of insert sequences collectively corresponds to at least 20% of a harmful biological sequence in the database; and
iii) automatically generating an alert if at least 20% of the harmful biological sequence is detected.

17. The system of claim 16, wherein if no alert is generated, then one or more polynucleotides are synthesized, wherein the one or more polynucleotides encode for the plurality of biological sequences.

18. The system of claim 16, further comprising receiving instructions for changing the vector and at least two of the plurality of insert sequences corresponding to at least 20% of the harmful biological sequence to remove the harmful biological sequence.

19. The system of claim 16, wherein the one or more design instructions are received at one or more time points.

20. The system of claim 16, wherein the one or more design instructions are received from different sources.

21. (canceled)

22. (canceled)

23. (canceled)

24. The system of claim 16, wherein the plurality of biological sequences are no more than 200 bases in length.

25. (canceled)

26. (canceled)

27. (canceled)

28. A method for providing enhanced polynucleotide synthesis comprising:
a) receiving one or more design instructions, wherein the design instructions comprise a plurality of biological sequences, wherein the plurality of biological sequences comprises a vector sequence, and a plurality of additional insert sequences;
b) automatically determining whether the vector and at least one of the plurality of insert sequences collectively corresponds to at least 20% of a harmful biological sequence in a database; and
c) automatically generating an alert if at least 20% of the harmful biological sequence is detected.

29. The method of claim 28, wherein the biological sequences are obtained from sequencing a physical nucleic acid or protein sample.

30. The method of claim 28, further comprising receiving instructions for changing the vector and at least two of the plurality of insert sequences corresponding to at least 20% of the harmful biological sequence to remove the harmful biological sequence.

31. The method of claim 28, further comprising wherein if no alert is generated, then one or more polynucleotides are synthesized, wherein the one or more polynucleotides encode for the plurality of biological sequences.

32. The computerized system of claim 1, wherein the harmful biological sequence encodes for a gene or gene fragment that, when translated or transcribed, results in a pathogenic product.

33. The computerized system of claim 32, wherein the gene is from a bacterial or a viral genome.

34. The method of claim 13, wherein the harmful biological sequence encodes for a gene or gene fragment that, when translated or transcribed, results in a pathogenic product.

35. The method of claim 34, wherein the gene is from a bacterial or a viral genome.


SEQUENCE LISTING (1 sequence)

SEQ ID NO: 1 - protein, 571 aa, Avian avulavirus 1
MDRAVNRVVLENEEREAKNTWRLVFRIAVLLLMVMILAISAAALAYSMEASTPHDLAGISTVISKTEDKVTSLLSSSQDVIDRIYKQVALESPLALLNTESVIMNAITSLSYQINGAKNSSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRIFFSTLRSINLDDTQNRKSCSVSATPLGCDMLCSKVTGTEEEDYKSVAPTSMVHGRLGFDGQYHEKDLDTTVLFKDWVANYPGVGGGSFINGRVWFPVYGGLKPNSPSDTAQEGKYVIYKRHNNTCPDKQDYQIRMAKSSYKPGRFGGKRIQQAILSIKVSTSLGKDPVLTIPPNTITLMGAEGRILTVGTSHFLYQRGSSYFSPALLYPMTVNNKTATLHSPYMFNAFTRPGSVPCQASARCPNSCITGVYTDPYPLIFYRNHTLRGVFGTMLDDEQARLNPVSAVFDNISRSRVTRVSSSSTKAAYTTSTCFKVVKTNKAYCLSIAEISNTLFGEFRIVPLLVEILKDDRV